(19) United States
(12) Patent Application Publication
(10) Pub. No.: US 2008/0044366 A1
(43) Pub. Date: Feb. 21, 2008

(54) COMPOSITIONS FOR APPLICATION TO THE SKIN, TO THE LIPS, TO THE NAILS, AND/OR TO HAIR

(75) Inventor: Christophe DUMOUSSEAUX, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/770,177

(22) Filed: Jun. 28, 2007

Related U.S. Application Data
(63) Continuation of application No. 11/100,509, filed on Apr. 7, 2005, now abandoned.
(60) Provisional application No. 60/564,966, filed on Apr. 26, 2004.

(30) Foreign Application Priority Data
Apr. 8, 2004 (FR) 04 50714

(51) Int. Cl. A61K 8/19; A61Q 1/02; A61Q 1/06; A61Q 3/02

(52) U.S. Cl. 424/61; 424/63; 424/64; 424/70.7

(57) ABSTRACT

The present disclosure relates to compositions comprising particles of at least one composite pigment, the particles comprising an inorganic core which is at least partially coated with at least one organic coloring substance, wherein the composition has a covering power ranging from 1 to 25, and wherein the at least partially coated inorganic core is present in an amount sufficient for the saturation C* of the composition to range from 25 to 100.

This non provisional application claims the benefit of French Application No. 04/50714 filed on Apr. 8, 2004 and U.S. Provisional Application No. 60/564,966 filed on Apr. 26, 2004, the contents of both of which are incorporated herein by reference.

The present disclosure relates to cosmetic compositions for application to the skin, including the mucous membranes, such as to the lips, to the nails, or to hair, for instance, the eyelashes, the eyebrows, and head hair.

It is known to use pigments for coloring a cosmetic composition, for example, pigments having particles that have an average size ranging from 200 nanometers (nm) to 100 nm. The compositions that include such pigments can be colored and it is possible to vary the transparency of the color and also the saturation C* thereof by varying the concentration of the pigment. In order to obtain cosmetic compositions that are not very opaque, which can be desirable, in order to confer a natural appearance on make-up, for example, or in order to avoid hiding point-like sparkle optical effects due to reflective fillers, the concentration of the pigment is reduced, which may have the drawback of simultaneously reducing the intensity of the coloring of the composition.

Sometimes inorganic pigments are used, for example, titanium oxide or iron oxide in the form of nanoparticles. But such pigments may not allow a large number of colors to be obtained, and the colors that are obtained may be relatively unsaturated.

In order to obtain a composition that is both colored and transparent, it is known to use soluble coloring agents. However, not many soluble coloring agents are authorized for use in cosmetics, and moreover they can have the drawback of transferring onto the made-up surface and of staining said surface.

Therefore, a need exists to have a cosmetic composition available that is relatively transparent or translucent, and that is of a color that is sufficiently saturated.

Accordingly, the present disclosure relates to a composition with a covering power that can range from 1 to 25, for instance, lower than 25, the composition being for application to the skin, to the lips, to the nails, and/or to hair, and comprising particles of at least one composite pigment, wherein the particles comprise an inorganic core, which is at least partially coated with at least one organic coloring substance.

The at least partially coated inorganic core can be present in an amount sufficient for the saturation C* of the composition to range for example from 25 to 100, for instance ranging from 30 to 100.

Thus, the present disclosure makes it possible to obtain compositions that are both relatively transparent or translucent and saturated.

For example, the composition may present a covering power ranging from 5 to 24.9, such as from 10 to 24.9, for example from 15 to 24.9.

The at least one composite pigment may be present in the composition in an amount ranging from 0.05% to 10%, for instance from 0.1% to 8% by weight, such as from 0.1% to 5% by weight, relative to the total weight of the composition, for example ranging from 0.1% to 3%, or from 0.5% to 3%.

A suitable tint may be obtained in a variety of manners, for example by mixing the composite pigments of the present disclosure, said pigments having different colors, and/or by the presence of a plurality of organic coloring substances when coating of the cores of the at least one composite pigment, said organic coloring substances being mixed or being in the respective layers of the coating.

As used herein, the term “at least partially coating” is understood to mean coating all or a portion of the inorganic core.

The composition of the present disclosure may include a physiologically acceptable medium. As used herein, the term “physiologically acceptable medium” is understood to mean a non toxic medium that can be applied to the skin, to the lips, to the nails, or to hair of human beings, for instance a cosmetic medium. The physiologically acceptable medium will be adapted to the nature of the surface onto which the composition is to be applied, and to the form in which the composition is intended to be packaged, such as a solid or a fluid at ambient temperature and atmospheric pressure.

As used herein, the term “cosmetic composition” is understood to mean a composition as defined in European Council Directive 93/35/EEC dated 14 Jun. 1993.

When evaluating a liquid or pasty composition, a sample of the composition to be studied is introduced into a small metal cup that is about 1 centimeter (cm) deep. A blade of quartz that is 1 millimeter (mm) thick is pressed against the composition, taking care to avoid air bubbles before taking the measurement.

When evaluating a composition in powder form, the powder sample is compacted at a pressure of 10 megapascals (MPa) in a small metal cup that is about 1 cm deep. A blade of quartz that is 1 mm thick is pressed against the composition before taking the measurement.

When evaluating a stick, the formulation is initially ground down so as to obtain a viscous paste. The composition is then poured into a mold made of quartz that that has a flat bottom and is about 2 cm deep.

The L*, a*, b* chromaticity coordinates of the composition in the CIE L*a*b* space are measured by means of a MINOLTA CM-508d spectrocolorimeter, under a D65 illuminant, with a specular component included, and in d/8 mode. The saturation C* of the composition is calculated by means of the formula C*=[(a*)²+(b*)²]^{1/2}.

For a powder, 50 parts by weight of the powder are ground down with 50 parts by weight of dimethicon (DOW CORNING DC 200 Fluid 5CST) so as to obtain a viscous paste.

Then, the formulation is spread to a thickness of 30 micrometers (μm) on an Erichsen contrast card, type 24/5, that presents a black background and a white background, and the (X, Y, Z) chromaticity coordinates are measured by means of a CR-300 colorimeter.

The composition is similarly spread on other contrast cards and three measurements are taken for each card. The average of these nine measurements is then calculated.

The covering power is equal to 100×Yn/Yb, where Yn is the average value of Y on a black background and Yb is the average value of Y on a white background. A covering power of 100 corresponds to a formulation that is completely opaque.

In one embodiment of the present disclosure, the at least one composite pigment is not an interference pigment. As used herein, the term “interference pigment” is understood to mean, for example, a pigment comprising a superposition of layers of constant thickness of materials selected to produce optical interferences. An example of an interference pigment is disclosed in U.S. Pat. No. 6,428,773.

In another embodiment of the present disclosure, the at least one organic coloring substance is not melanin.

The saturation C* of the at least one composite pigment may be greater than or equal to 30, measured according to the following protocol. Protocol for measuring the saturation C* of the at least one composite pigment:

The color values a* and b* in the CIE L*a*b* colorspace of the at least one composite pigment are measured as follows.

The composite pigment in a raw state is compacted in a rectangular cup having dimensions of 2×1.5 cm and a depth of 3 mm, by applying a pressure of 100 bars.

The a* and b* values of the compacted pigment are measured with a Minolta 3700d spectrophotometer, in mode specular excluded under illuminant D65 and medium aperture. The saturation is computed as C*=(a*²+b*²)^{1/2}.

The composition can be a glossy composition. The composition can exhibit, for example when it is a liquid, a mean gloss T₀h greater than or equal to 30, for instance, 35, or 40, or 45, or 50, or 55, or 60, or 65, or 70 or such as 75 out of 100, for example, when the composition is intended to be applied on the lips.

The mean gloss T₀h can be measured according to the following protocol:

Protocol for Measuring the Mean Gloss T₀h:

As used herein, the term “mean gloss” is understood to mean the gloss as can be conventionally measured using a glossmeter, by the following method:

On a constrat card of the brand BYK Gardner and of reference Prüfkarten, Art. 2853, previously fixed on a glass plate of 1 mm of thickness, a layer that is 25 μm thick of the composition is sprayed using an automatic sprayer (Bar coater, Sheen).

The layer covers at least the black background of the card.

When the composition is a solid, one melts it if necessary on the card after having spread it so that it covers the black background.

As soon as the composition is spread, one measures the mean gloss at an angle of 60°, on the black background of the card using a BYK GARDNER Brand glossmetter of reference microTRI-GLOSS.

Four constrast card are prepared this way to measure the mean gloss of the composition and to compute the average of the four values, T₀h.

The measure is deemed to be correct when the standard deviation of the four values is less or equal to 3%.

The mean gloss T₅h after five hours can also be measured.

The contrast card is left for five hours on a thermostated plate at a temperature of 30° C.

After five hours, the contrast card is removed from the thermostated plate and allowed to return to room temperature. The mean gloss is again measured at 600, called mean gloss T₅h, by averaging four values.

In one embodiment of the present disclosure, when gloss is sought, the mean gloss T₀h of the composition at 600 can be greater than or equal to 45, for instance greater than 50, or 60, or 65, or 70, such as 75.

The mean gloss T₅h measured at 600 can be greater than or equal to 35, or 40, or 45, or 50, or 55, or 60, or 65 or 70, and can be, for example, 75, out of 100.

The particulate phase in the composition can be present in an amount of greater than or equal to 5%, for example greater than or equal to 7.5%, for instance greater than or equal to 10%, such as greater than or equal to 15%, 20% or 30%.

Without being bound by theory, the presence of composite pigments in the compositions of the present disclosure can enable the compositions to have a relatively high gloss notwithstanding a relatively high amount of particulate phase.

Protocol for Measuring the Amount of Particulate Phase:

A Soxhlet extractor comprising a cartridge, a fat flask, a flask heater and a condensor is used.

The Soxhlet extractor cartridge is first regenerated by boiling about 80 ml of toluene in the fat flask so that the cycles lasts about one half-hour. The cartridge is dried in an oven one night and then in the dessicator.

A PTFE membrane having a known weight T₁ is folded in a cone and inserted into the cartridge. Precisely 0.75 g (m) of the composition is weighed in the membrane and the latter is folded in the cartridge so that it is well closed.

The cartridge is put in the Soxhlet extractor after having introduced a small perforated flask, serving to maintain the top of the cartridge a little above the elbow of the Soxhlet extractor to prevent the level of toluene to exceed the top end of the cartridge and to prevent the displacement of the composition.

80 ml of toluene are added in the fat flask.

The condenser is started and heats the fat flask so that the toluene boils (boiling point 100.6° C.) with reflux for four hours.

The vapors of toluene have to condense at the first ball of the condenser and the condensation must not be too fast. The condenser is then cooled and switched off.

The cartridge is dried in the oven for two days and left in the dessicator for at least two hours, and the dry cartridge is weighed (T₂) immediately after leaving the dessicator.

The test is made on two samples at least.

The amount of particulate phase, i.e. materials not soluble in hot toluene, is given by Q=((T₂−T₁)/m)×100.

### BRIEF DESCRIPTION OF THE DRAWINGS

FIG. 1: The curves corresponding to the total transmission measurements for the compositions of Examples 1 and 2 and for the Comparative Example are reproduced in FIG. 1.

The at least one composite pigment of the present disclosure can be composed of particles comprising an inorganic core, which is at least partially coated with at least one organic coloring substance.

At least one binder may, for example, contribute to fixing the at least one organic coloring substance onto the inorganic core.

The particles of the at least one composite pigment can have a variety of forms. For instance, the particles can be in the form of flakes or they may be globular, such as spherical, and may be hollow or solid. As used herein, the term “in the form of flakes” is understood to mean particles for which the ratio of the largest dimension to the thickness is 5 or more.

The at least one composite pigment of the present disclosure can, for example, have a specific surface area ranging from 1 m²/g (square meters/gram) to 1,000 m²/g, for instance, ranging from 10 m²/g to 600 m²/g, and such as ranging from 20 m²/g to 400 m²/g. The specific surface area is the value measured using the BET (Brunauer-Emmett-Teller) method.

The composition can comprise at least one composite pigment solely of the kind defined above or, in one embodiment of the present disclosure, it may comprise at least one other composite pigment, as well as pigments having a non composite structure, such as mineral pigments, interference pigments, lakes or other organic pigments. For example, the composition can be free of uncoated TiO₂ particles.

The inorganic core may have any form that is suitable for fixing particles of organic coloring substance, for example, non-limiting mention may be made of the forms spherical, globular, granular, polyhedral, acicular, spindle-shaped, flattened in the form of a flake, a rice grain, or a scale, and a combination of these forms.

For example, the ratio of the largest dimension of the inorganic core to its smallest dimension can range from 1 to 50.

The inorganic core can have a mean size ranging from 1 nm to 100 nm, for instance, ranging from 5 nm to 75 nm, for example ranging from 10 nm to 50 nm.

As used herein, the term “mean size” is understood to mean the dimension given by the statistical grain size distribution curve at 50% population, termed D50. The mean size may be a number average determined by image analysis (electron microscopy).

The inorganic core can have a refractive index greater than or equal to 2, such as greater than or equal to 2.1, for example greater than or equal to 2.2.

Among the material that the inorganic core can be formed from, non-limiting mention can be made of metallic salts and metal oxides, such as oxides of titanium, zirconium, cerium, zinc, iron, iron blue, aluminum, and chromium, aluminas, glasses, ceramics, graphite, silicas, silicates, for instance aluminosilicates and borosilicates, synthetic mica, and mixtures thereof.

Further non-limiting mention can be made of oxides of titanium, such as TiO₂, of iron, such as Fe₂O₃, of cerium, zinc, and aluminum, silicas and silicates, for instance aluminosilicates and borosilicates, which can also be used.

The inorganic core can have a specific surface area, measured using the BET method, ranging from 1 m²/g to 1,000 m²/g, for instance, ranging from 10 m²/g to 600 m²/g, for example ranging from 20 m²/g to 400 m²/g.

The inorganic core can be colored if desired.

The inorganic core in the at least one composite pigment can be present in an amount greater than or equal to 50% by weight, relative to the total weight of the composite pigment, for example in an amount ranging from 50% to 70%, such as from 60% to 70% by weight, relative to the total weight of the composition.

The at least one organic coloring substance can, for example, comprise at least one organic pigment, for example at least one organic lake. The at least one organic coloring substance may, for example, be chosen from compounds that are insoluble in the physiologically acceptable medium of the composition.

The at least one organic coloring substance may, for example, comprise pigments, for example organic lakes or other coloring substances, that can be chosen from:
cochineal carmine;
the organic pigments of azo, anthraquinone, indigo, xanthene, pyrene, quinoline, triphenylmethane, or fluorane dyes;
organic lakes or organic insoluble salts of sodium, potassium, calcium, barium, aluminum, zirconium, strontium, titanium, or of acid dyes such as azo, anthraquinone, indigo, xanthene, pyrene, quinoline, triphenylmethane, or fluorine dyes, which dyes may comprise at least one carboxylic or sulfonic acid group.

Among the organic pigments that may be used, non-limiting mention may be made of: D&C Blue No.4, D&C Brown No.1, D&C Green No.5, D&C Green No.6, D&C Orange No.4, D&C Orange No.5, D&C Orange No.10, D&C Orange No.11, D&C Red No.6, D&C Red No.7, D&C Red No.17, D&C Red No.21, D&C Red No.22, D&C Red No.27, D&C Red No.28, D&C Red No.30, D&C Red No.31, D&C Red No.33, D&C Red No.34, D&C Red No.36, D&C Violet No.2, D&C Yellow No.7, D&C Yellow No.8, D&C Yellow No.10, D&C Yellow No.11, FD&C Blue No.1, FD&C Green No.3, FD&C Red No.40, FD&C Yellow No.5, FD&C Yellow No.6.

The at least one organic coloring substance may comprise at one organic lake supported by an organic support such as colophane or aluminum benzoate, for example.

Among the organic lakes that may be used, non-limiting mention may be made of: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, and FD&C Yellow No. 6 Aluminum lake.

The chemical compounds corresponding to each of the organic coloring substances listed above are mentioned in the book entitled International Cosmetic Ingredient Dictionary and Handbook, 1997 edition, pages 371 to 386 and 524 to 528, published by “The Cosmetic, Toiletry, and Fragrance Association,” the contents of which are hereby incorporated by reference.

The at least one organic coloring substance can be present in an amount ranging from 10 parts to 500 parts by weight per 100 parts of inorganic core, for instance ranging from 20 parts to 250 parts by weight, for example ranging from 40 parts to 125 parts by weight per 100 parts of inorganic core.

The at least one organic coloring substance in the at least one composite pigment can be present in an amount greater than or equal to 30% by weight, relative to the total weight of the at least one composite pigment, for example ranging from 30% to 50%, such as from 30% to 40% by weight, relative to the total weight of the at least one composite pigment.

The at least one binder can be of any type, provided that it allows the organic coloring substance to adhere to the surface of the inorganic core.

For instance, the at least one binder can be chosen from silicone compounds, polymeric, oligomeric and similar compounds, such as from organosilanes, fluoroalkylated organosilanes and polysiloxanes, for example polymethylhydrogen siloxane, as well as a variety of coupling agents such as coupling agents based on silanes, titanates, aluminates, and zirconates. Among the silicone compounds that can be used, non-limiting mention can be made of:
organosilanes (1) obtained from alkoxysilanes;
polysiloxanes (2) which may optionally be modified, non-limiting examples of which include:
   modified polysiloxanes (2A) comprising at least one radical chosen for instance from polyethers, polyesters and epoxy compounds (termed “modified polysiloxanes” below);
   polysiloxanes (2B) carrying, on one silicon atom located at the end of the polymer, at least one functional group chosen from, by way of non-limiting example: carboxylic acids, alcohols, and hydroxyl groups; and
fluoroalkylated organosilane compounds (3) obtained from fluoroalkylsilanes.

The organosilane compounds (I) may be obtained from alkoxysilane compounds chosen from those of formula (I):
R¹ₐSiX₄₋ₐ (I)
wherein:

R¹ is chosen from C₆H₅—, (CH₃)₂CH—CH_{2b+1}— and CbH_{2b+1}— groups (in which b ranges from 1 to 18);

X is chosen from CH₃O— and C₂H₅O— groups; and

a ranges from 0 to 3.

Non-limiting examples of alkoxysilane compounds may include alkoxysilanes chosen from: methyltriethoxysilane, dimethyldiethoxysilane, phenyltriethoxysilane, diphenyldiethoxysilane, methyltrimethoxysilane, dimethyldimethoxysilane, phenyltrimethoxysilane, diphenyldimethoxysilane, isobutyltrimethoxysilane, decyltrimethoxysilane. For example, in one embodiment of the present disclosure, the at least one alkoxysilane compound is chosen from methyltriethoxysilane, phenyltriethoxysilane, methyltrimethoxysilane, dimethyldimethoxysilane, isobutyltrimethoxysilane, such as from methyltriethoxysilane, methyltrimethoxysilane, and phenyltriethoxysi lane.

The polysiloxanes (2), for example, can be chosen from those of formula (II): 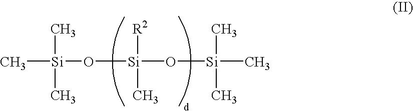
wherein R² is chosen from a hydrogen atom and CH₃ groups and d ranges 15 to 450.

In one embodiment of the present disclosure, R² is a hydrogen atom.

The modified polysiloxanes (2A) can be, for example,

(a¹) modified polysiloxanes carrying polyethers, chosen from those of formula (III): 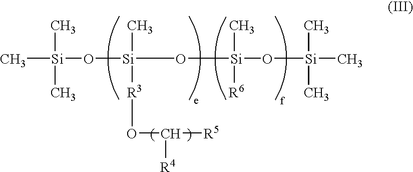
wherein R³ is chosen from —(CH₂)ₕ— groups; R⁴ is chosen from —(CH₂)ᵢ—CH₃ groups; R⁵ is chosen from —OH, —COOH, —CH═CH₂, —C(CH₃)═CH₂ and —(CH₂)ⱼ—CH₃ groups; R⁶ is chosen from —(CH₂)ₖ—CH₃ groups; q and h, independently from each other, range from 1 to 15; j and k, independently from each other, range from 0 to 15; e ranges from 1 to 50, and f ranges from 1 to 300;
(a²) modified polysiloxanes carrying polyesters, represented by formula (IV): 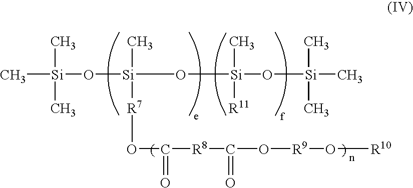
   wherein R⁷, R⁸₇ and R⁹, which may be identical or different, are chosen from —(CH₂)_{q}— groups; R¹⁰ is chosen from —OH, —COOH, —CH═CH₂, —C(CH₃)═CH₂ and —(CH₂)ᵣ—CH₃ groups; R¹¹ is chosen from —(CH₂)ₛ— CH₃ groups; n and q, which may be identical or different, range from 1 to 15, r and s, which may be identical or different, range from 0 to 15; e ranges from 1 to 50, and f ranges from 1 to 300; and
(a³) modified polysiloxanes carrying epoxy radicals chosen from those of formula (V): 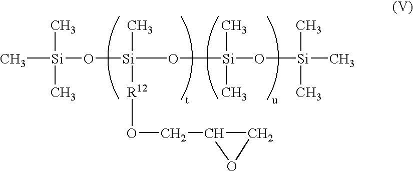
   wherein R¹² is chosen from —(CH₂)ᵥ— groups; v ranges from 1 to 15; t ranges from 1 to 50, and u ranges from 1 to 300; or mixtures thereof.

In one embodiment of the present disclosure, the polysiloxanes (2A) are modified polysiloxanes carrying polyethers chosen from those of formula (III).

Polysiloxanes modified at the terminal portion (2B) can be chosen from those of formula (VI): 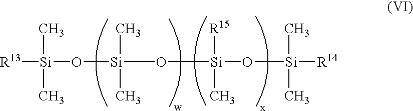
wherein R¹³ and R¹⁴, which may be identical or different, are chosen from OH, R¹⁶—OH, and R¹⁷—COOH groups; R¹⁵ is chosen from —CH₃ and —C₆H₅ groups; R¹⁶ and R¹⁷ are chosen from —(CH₂)_{y}— groups; y ranges from 1 to 15; w ranges from 1 to 200; and x ranges from 0 to 100.

In one embodiment of the present disclosure, at least one of R¹⁶ and/or R¹⁷ carry a carboxylic acid group on at least one terminal silicon atom.

Fluoroalkylated organosilane compounds (3) can be chosen from fluoroalkylsilanes of formula (V11):
CF₃(CF₂)_{z}CH₂CH₂(R¹⁸)ₐSiX₄₋ₐ (VII)
wherein:

R¹⁸ is chosen from CH₃—, C₂H₅—, CH₃O—, and C₂H₅O— groups;

X is chosen from CH₃O— and C₂H₅O— groups;

z ranges from 0 to 15 and a ranges from 0 to 3.

Among the fluoroalkylsilanes that may be used, non limiting mention may be made of: trifluoropropyltrimethoxysilane, tridecafluorooctyltrimethoxysilane, heptadecafluorodecyltrimethoxysilane, heptadecafluorodecylmethyldimethoxysilane, trifluoropropyltriethoxysilane, tridecafluorooctyltriethoxysilane, heptadecafluorodecyltriethoxysilane, heptadecafluorodecylmethyldiethoxysilane and the like. In one embodiment, the fluoroalkylsilanes are chosen from those such as trifluoropropyltrimethoxysilane, tridecafluorooctyltrimethoxysilane and heptadecafluorodecyltrimethoxysilane, and in another embodiment, the fluoroalkylsilanes are chosen from trifluoropropyl trimethoxysilane and tridecafluorooctyltrimethoxysilane.

Non-limiting examples of the silane-based coupling agents that can beused include vinyltrimethoxysilane, vinyltriethoxysilane, γ-aminopropyl-triethoxysilane, γ-glycidoxypropyltrimethoxysilane, γ-mercaptopropyltrimethoxysilane, γ-methacryloxypropyltrimethoxysilane, N-β(aminoethyl)-γ-aminopropyltrimethoxysilane, γ-glycidoxypropylmethyldimethoxysilane, and γ-chloropropyltrimethoxysilane.

Non-limiting examples of the titanate-based coupling agents that can be used include isopropylstearoyl titanate, isopropyltris(dioctylpyrophosphate) titanate, isopropyltri(N-aminoethyl-aminoethyl)titanate, tetraoctylbis(ditridecylphosphate) titanate, tetra(2,2-diaryloxymethyl-1-butyl)bis(ditridecyl)phosphate titanate, bis(dioctylpyrophosphate)oxyacetate titanate, and bis(dioctylpyrophosphate)ethylene titanate.

Non-limiting examples of the aluminate-based coupling agents that can be used include acetoalkoxyaluminum diisopropylate, aluminum diisopropoxymonoethylacetoacetate, aluminum trisethylacetoacetate, and aluminum trisacetylacetonate.

Non-limiting examples of the zirconate-based coupling agents that can be used include zirconium tetrakisacetylacetonate, zirconium dibutoxybisacetylacetonate, zirconium tetrakisethylacetoacetate, zirconium tributoxymonoethylacetoacetate, and zirconium tributoxyacetylacetonate.

The compounds acting as a binder can have a molar mass ranging from 300 to 100 000.

To obtain a layer which uniformly coats the inorganic cores, the binder may be, for example, in the liquid state or may be soluble in water or other solvents.

The at least one binder may be present in an amount ranging from 0.01% to 15%, for instance from 0.02% to 12.5%, and such as from 0.03% to 10% by weight (calculated with respect to C or Si) relative to the weight of particles comprising the core and the binder. Further details regarding the calculation of the relative quantity of binder can be found in European Patent Application No. EP 1 184 426 A2.

The at least one binder can be present in an amount less than or equal to 5%, such as less than or equal to 3% by weight, relative to the total weight of the composite pigment.

The at least one composite pigment can, for example, be produced using one of the processes described in European Patent Application Nos. EP 1 184 426 and EP 1 217 046, the contents of which are hereby incorporated by reference. In one embodiment of the present disclosure, the at least one composite pigment is produced according to the process described in European Patent No. EP 1 184 426.

In one embodiment of the present disclosure, the particles intended to constitute the inorganic core are first mixed with the at least one binder.

So that the at least one binder can adhere uniformly to the surface of the inorganic core, the particles may, for example, pass initially through a mill to disaggregate them.

The mixing and agitation conditions are chosen so that the core is uniformly coated with binder. Such conditions may be controlled so that the linear load is ranges from 19.6 N/cm (newtons/centimeter) to 19,160 N/cm, for instance, ranging from 98 N/cm to 14,170 N/cm and such as ranging from 147 N/cm to 980 N/cm; the period of treatment time ranges frp, 5 minutes to 24 hours, for instance, from 10 minutes to 20 hours; the rotation rate can be ranging from 2 rpm (revolutions per minute) to 1,000 rpm, such as ranging from 5 rpm to 1,000 rpm, and for example, ranging from 10 rpm to 800 rpm.

After coating the inorganic core with binder, the at least one organic coloring substance is added and mixed with agitation so that it at least adheres partially to the layer of binder.

Examples of addition methods include continuous addition in large quantities, or in small quantities.

Mixing and agitation, whether of the inorganic cores with the binder or of the at least one organic coloring substance with the inorganic cores coated with binder, can be carried out using an apparatus which can apply a sharp shearing and/or compressive force to the mixture of powders. Examples of apparatus of that type are roller mixers, blade mixers, and the like. In one embodiment of the present disclosure, a roller mixer is used. A list of suitable apparatus is given in European Application No. EP 1 184 426 A2.

Another method for manufacturing the at least one composite pigment has been described in Japanese Patent No. JP 3286463, which discloses a solution precipitation process. The at least one organic coloring substance is dissolved in ethanol and the inorganic cores are then dispersed in said ethanolic solution. An aqueous alkaline solution of sodium or potassium carbonate is then slowly added to these mixtures and finally, an ethanolic calcium chloride solution is slowly added, with constant agitation.

The composition may include at least one aqueous or organic solvent.

When the composition includes at least one organic solvent, the solvent can be present in a total amount ranging from 0.1% to 99% by weight, relative to the total composition weight.

In general, the quantity of the at least one solvent, such as the at least one organic solvent, will depend on the nature of the surface onto which the composition is intended to be applied.

In the case of a nail polish, for example, the at least one organic solvent may be present in the composition in an amount ranging from 30% to 99% by weight, such as from 60% to 90% by weight, for example, relative to the total composition weight.

The at least one organic solvent can be chosen from:
ketones which are liquid at ambient temperature, such as methylethylketone, methylisobutylketone, diisobutylketone, isophorone, cyclohexanone, or acetone;
alcohols which are liquid at ambient temperature, such as ethanol, isopropanol, diacetone alcohol, 2-butoxyethanol, or cyclohexanol;
glycols which are liquid at ambient temperature, such as ethylene glycol, propylene glycol, pentylene glycol or glycerol;
propylene glycol ethers which are liquid at ambient temperature, such as propylene glycol monomethyl ether, the acetate of propylene glycol monomethyl ether, or dipropylene glycol mono n-butyl ether;
short chain esters (comprising a total of 3 to 8 carbon atoms), such as ethyl acetate, methyl acetate, propyl acetate, n-butyl acetate, or isopentyl acetate; and
alkanes which are liquid at ambient temperature, such as decane, heptane, dodecane, or cyclohexane.

The composition may also comprise water or a mixture of water and hydrophilic organic solvents which are routinely used in cosmetics, such as alcohols, for instance, linear or branched lower monoalcohols comprising from 2 to 5 carbon atoms, such as ethanol, isopropanol or n-propanol, polyols such as glycerine, diglycerine, propylene glycol, sorbitol, penthylene glycol, or polyethylene glycols. The composition may also comprise hydrophilic C₂ ethers and C₂-C₄ aldehydes. The water or mixture of water and hydrophilic organic solvents may be present in the composition in an amount ranging from 0% to 90%, for instance, from 0.1% to 90% by weight, such as from 0% to 60% by weight, and from 0.1% to 60% by weight, relative to the total composition weight.

When it is to be applied to the lips, for example, the composition may also comprise an oily phase, for instance, at least one fat that is liquid at ambient temperature (25° C.) and/or a fat that is solid at ambient temperature, such as waxes, pasty fats, gums, and mixtures thereof. The oily phase may also comprise lipophilic organic solvents.

The composition may, for example, have a continuous oily phase which may comprise less than 5% water, for instance, less than 1% water relative to its total weight, for example, it may be in the anhydrous form.

Among the fats that are liquid at ambient temperature, also called “oils,” that may be used, non-limiting mention may be made of hydrocarbon-comprising vegetable oils such as liquid fatty acid triglycerides comprising from 4 to 10 carbon atoms, for example heptanoic or octanoic acid triglycerides, or sunflower, corn, soybean, grapeseed, sesame seed, apricot kernel, macadamia nut, castor, or avocado stone oil, caprylic/capric acid triglycerides, jojoba oil, shea nut butter oil, lanolin, acetylated lanolin; linear or branched hydrocarbons of mineral or synthetic origin, such as paraffin oils and their derivatives, petroleum jelly, polydecenes, hydrogenated polyisobutene such as Parleam; synthesized esters and ethers, for instance, fatty acids such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyidodecyl hydroxystearate, diisostearylmalate, triisocetyl citrate, fatty alcohol heptanoates, octanoates or decanoates; isononyl isonanoate, isopropyl lanolate, tridecyl trimellilate, diisostearyl malate; polyol esters such as propylene glycol dioctanoate, neopentylglycol diheptanoate, diethyleneglycol diisononanoate; and pentaerythritol esters; fatty alcohols comprising from 12 to 26 carbon atoms, such as octyidodecanol, 2-butyloctanol, 2-hexyldecanol, 2-undecylpentadecanol or oleic alcohol; partially hydrocarbonated and/or siliconized fluorinated oils; silicone oils such as volatile or non volatile, linear or cyclic polymethylsiloxanes (PDMS) which may be liquid or pasty at ambient temperature, such as cyclomethicones or dimethicones, optionally comprising a phenyl group, such as phenyl trimethicones, phenyltrimethylsiloxydiphenyl siloxanes, diphenylmethyldimethyl-trisiloxanes, diphenyl dimethicones, phenyl dimethicones, polymethylphenylsiloxanes; and mixtures thereof. The oils may be present in an amount ranging from 0.01% to 90%, such as from 0.1% to 85% by weight, relative to the total composition weight.

Pasty fats are generally hydrocarbon-comprising compounds with a melting point ranging from 25° C. to 60° C., for example ranging from 30° C. to 45° C. and/or with a hardness ranging from 0.001 MPa (megapascals) to 0.5 MPa, for instance ranging from 0.005 MPa to 0.4 MPa, such as lanolins and derivatives thereof.

Waxes may be solid at ambient temperature (25° C.) with a reversible solid/liquid change of state, with a melting point of greater than 30° C. and up to 200° C., a hardness of greater than 0.5 MPa, and with an anisotropic crystalline organization in the solid state. For example, the waxes may have a melting point of more than 25° C., such as more than 45° C. The waxes may be hydrocarbon-comprising, fluorinated and/or siliconized and may be of animal, mineral, vegetable and/or synthetic origin. Among the suitable waxes that may be used, non-limiting mention can be made of beeswax, carnauba wax or candellila wax, paraffin, microcrystalline waxes, ceresin, or ozokerite; synthetic waxes such as polyethylene or Fischer-Tropsch waxes or silicone waxes such as alkyl or alkoxy-dimethicone comprising from 16 to 45 carbon atoms. The composition may comprise from 0% to 50% by weight of waxes relative to the total composition weight, such as from 1% to 30% by weight.

Suitable gums that may be used include high molecular weight polydimethylsiloxanes (PDMS) or cellulose gums or polysaccharides.

In one embodiment of the present disclosure, the composition can comprise at least one oil with a molar mass ranging from 650 g/mol to 10,000 g/mol. As used herein, the term “oil” is understood to mean a nonaqueous compound which is immiscible with water, and which is liquid at room temperature (25° C.) and atmospheric pressure (760 mmHg). For example, the at least one oil can have a molar mass ranging from 650 g/mol to 10,000 g/mol, such as ranging from 750 g/mol to 7,500 g/mol.

Among the oils having a molar mass ranging from 650 g/mol to 10,000 g/mol that may be used, non-limiting meniton can be made of:
lipophilic polymers such as:
   polybutylenes such as INDOPOL H-100 (having a molar mass or MM=965 g/mol), INDOPOL H-300 (MM=1 340 g/mol), INDOPOL H-1500 (MM=2 160 g/mol) which are marketed or manufactured by the company AMOCO,
   hydrogenated polyisobutylenes such as PANALANE H-300 E which are marketed or manufactured by the company AMOCO (M=1 340 g/mol), VISEAL 20000 marketed or manufactured by the company SYNTEAL (MM=6 000 g/mol), REWOPAL PIB 1000 marketed or manufactured by the company WITCO (MM=1 000 g/mol),
   polydecenes and hydrogenated polydecenes such as PURESYN 10 (MM=723 g/mol), PURESYN 150 (MM=9 200 g/mol) which are marketed or manufactured by the company MOBIL CHEMICALS,
   copolymers of vinypyrrolidone such as: the vinylpyrrolidone/1-hexadecene copolymer, ANTARON V-216, marketed or manufactured by the company ISP (MM=7 300 g/mol),
esters such as:
   linear fatty acid esters having a total number of carbons ranging from 35 to 70 such as pentaerythrityl tetrapelargonate (MM=697.05 g/mol),
   hydroxylated esters such as 2-polyglyceryl triisostearate (MM=965.58 g/mol),
   aromatic esters such as tridecyl trimellitate (MM=757.19 g/mol),
   C₂₄-C₂₈ branched fatty acid or fatty alcohol esters such as those described in Application EP-A-0 955 039, for example, triisoarachidyl citrate (MM=1033.76 g/mol), pentaerythrityl tetraisononanoate (MM=697.05 g/mol), glyceryl triisostearate (MM=891.51 g/mol), glyceryl tri(2-decyltetradecanoate) (MM=1143.98 g/mol), pentaerythrityl tetraisostearate (MM=1202.02 g/mol), polyglyceryl-2 tetraisostearate (MM=1232.04 g/mol) or alternatively pentaerythrityl tetra(2-decyltetradecanoate) (MM=1538.66 g/mol),
silicone oils such as phenylated silicone such as BELSIL PDM 1000 from the company WACKER (MM=9 000 g/mol),
polyesters and esters obtained from dimer diol, such as for example esters of dimer diol and fatty acid, and esters from dimer diol and dimer diacid. For example esters of dilinoeic acid and dilinoleic diol sold by NIPPON FINE CHEMICAL under the name LUSPLAN DD-DA5® et DD-DA7®. These oils are described in detail in U.S. Patent Application Publication No. U.S. 2004-0175338, the content of which is incorporated herein by reference,
oils of plant origin such as sesame oil (820.6 g/mol),
and mixtures thereof.

For example, when present, the oil having a molar mass ranging from 650 g/mol to 10,000 g/mol can be present in an amount ranging from 1% to 99%, for instance from 10% to 80%, such as from 5% to 70% by weight, relative to the total weight of the composition.

The composition may also, for example, comprise at least one film-forming polymer, for example, for a mascara or a nail polish composition. As used herein, the term “film-forming polymer” is understood to mean a polymer that can form, by itself or in the presence of an additional film-forming agent, a continuous film that adheres to a surface, such as to keratinous materials.

Among the suitable film-forming polymers that may be used in the compositions in accordance with the present disclosure, non-limiting mention may be made of synthetic polymers, of the radical or polycondensate type, natural polymers such as nitrocellulose or cellulose esters, and mixtures thereof.

Radical type film-forming polymers may for example, be vinyl polymers or copolymers, such as acrylic polymers.

Vinyl film-forming polymers may result from polymerizing monomers with an ethylenically unsaturated bond comprising at least one acid group and/or esters of said acid monomers and/or amides of said acid monomers, such as α, β-ethylenically unsaturated carboxylic acids, for example acrylic acid, methacrylic acid, crotonic acid, maleic acid, or itaconic acid. Vinyl film-forming polymers may also result from homopolymerizing or copolymerizing monomers chosen from vinyl esters such as vinyl acetate, vinyl neodecanoate, vinyl pivalate, vinyl benzoate, and vinyl t-butyl benzoate, and styrene monomers such as styrene and alpha-methyl styrene.

Non-limiting examples of film-forming polycondensates that may be mentioned include polyurethanes, polyesters, polyester amides, polyamides, and polyureas.

Polymers of natural origin, which may optionally be modified, may be chosen from shellac resin, gum sandarac, dammar resin, gum elemi, copal resin, cellulose polymers such as nitrocellulose, ethylcellulose, or nitrocellulose esters chosen, for example, from cellulose acetate, cellulose acetobutyrate, and cellulose acetopropionate, and mixtures thereof.

The at least one film-forming polymer may be present in the form of solid particles in an aqueous or oily dispersion, generally known as latexes or psuedolatexes. The at least one film-forming polymer can comprise at least one stable dispersions of generally spherical polymer particles of at least one polymer in a physiologically acceptable liquid oily phase. Said dispersions are generally termed polymer NADs (non aqueous dispersions), in contrast to latexes which are aqueous polymer dispersions. Said dispersions may be in the form of nanoparticles of polymers in stable dispersion in said oily phase. The nanoparticle size can range, for example, from 5 nm to 600 nm. Techniques for preparing said dispersions are well known to the skilled person.

Among the aqueous film-forming polymer dispersions that may be used, non-limiting mention can be made of acrylic dispersions sold under the trade names NEOCRYL XK-90®, NEOCRYL A-1070®, NEOCRYL A-1090®, NEOCRYL BT-62®, NEOCRYL A-1079®, NEOCRYL A-523® by AVECIA-NEORESINS, and DOW LATEX 432® by DOW CHEMICAL; DAITOSOL 5000 AD® by DAITO KASEI KOGYO; aqueous polyurethane dispersions sold under the trade names NEOREZ R-981® and NEOREZ R-974® by AVECIA-NEORESINS; AVALURE UR-405®, AVALURE UR-410®, AVALURE UR-425®, AVALURE UR-450®, SANCURE 875®, SANCURE 861®, SANCURE 878®, and SANCURE 2060® by GOODRICH; IMPRANIL 85® by BAYER; AQUAMERE H-1511® by HYDROMER; and sulfopolyesters sold under the trade mark Eastman AQ by Eastman Chemical Products.

The compositions of the present disclosure may also comprise at least one auxiliary film-forming agent which encourages the formation of a film with the film-forming polymer.

The compositions may also comprise at least one filler. As used herein, the term “illers” is understood to mean particles of any form which are insoluble in the composition medium regardless of the temperature at which the composition is manufactured. Said fillers primarily act to modify the rheology or texture of the composition. The nature and quantity of the solid substances are a function of the desired mechanical properties and textures. Non-limiting examples of fillers that may be mentioned include talc, mica, silica, kaolin, and sericite, and powders of polyamide, polyethylene, polytetrafluoroethylene, polymethylmethacrylate, or polyurethane, powdered starch, and silicone resin beads.

The compositions may further comprise at least one additional coloring substance which differs from the at least one composite pigment used in the present disclosure.

The at least one additional coloring substance can be chosen from mineral pigments, organic pigments, pearlescent pigments, liposoluble and hydrosoluble colorants.

The mineral pigments may be white or colored, and may optionally be coated. Non-limiting examples of mineral pigments include titanium dioxide, which may be surface treated; oxides of zirconium or cerium; and oxides of iron or of chromium; manganese violet; ultramarine blue; chromium hydrate; and iron blue. The mineral pigments may be present in an amount ranging from 0% to 40%, for instance, from 1% to 35% and such as from 2% to 25% of the total composition weight.

The pearlescent pigments can be chosen from white pearlescent pigments such as mica coated with titanium, and bismuth oxychloride, colored pearlescent pigments, such as titanium mica with iron oxides, titanium mica with in particular iron blue and chromium oxide, titanium mica with an organic pigment of the above-mentioned type, as well as the bismuth oxychloride-based pearlescent pigments. The pearlescent pigments, when present, may be present in an amount ranging from 0% to 20% of the total weight of the composition, for instance, from 0.1% to 15%.

Non-limiting examples of organic pigments that may be used include carbon black, D&C type pigments, and lakes based on carmine cochineal, barium, strontium, calcium, or aluminum.

Non-limiting examples of liposoluble colorants include Sudan red, D&C Red No. 17, D&C Green No. 6, β-carotene, soybean oil, Sudan brown, D&C Yellow No. 11, D&C Violet No. 2, D&C orange No. 5, and quinoline yellow.

Non-limiting examples of hydrosoluble colorants that may be used include beetroot juice and methylene blue.

The colorants, when present, may be present in an amount ranging from 0.1% to 20% by weight, relative to the weight of the composition, such as from 0.1% to 6% by weight.

The compositions may also comprise at least one cosmetic and/or dermatological active ingredient. Suitable cosmetic, dermatological, hygienic, or pharmaceutical active ingredients for use in the compositions of the present disclosure that may be mentioned in a non-limiting manner are moisturizing agents (polyols such as glycerine), vitamins (C, A, E, F, B, or PP), essential fatty acids, essential oils, ceramides, sphingolipids, liposoluble or nanoparticle sun screens, and specific skin treatment active ingredients (protective agents, antibacterials, anti-wrinkle agents, etc). The at least one active ingredient, when present, for example, can be present in an amount ranging from 0% to 20%, for instance from 0.001% to 15% by weight, relative to the total composition weight.

The cosmetic compositions may also optionally comprise at least one ingredient which is routinely used in cosmetics, such as thickeners, surfactants, oligo-elements, moisturizing agents, softeners, sequestrating agents, fragrances, alkalinizing or acidifying agents, preservatives, antioxidants, UV filters, and colorants.

Depending on the envisaged application, the composition of the present disclosure may comprise constituents which are conventionally used in the fields under consideration, and which are present in quantities appropriate to the desired dosage or “galenical” form.

The composition may be in a variety of forms, depending on its intended purpose. The composition may thus be in any galenical form which is normally used for topical application, for instance, in the anhydrous form, in the form of an oily or aqueous solution, an oily or aqueous gel, an oil-in-water emulsion, a water-in-oil emulsion, a wax-in-water or a water-in-wax emulsion, a multiple emulsion or a dispersion of oil in water due to vesicles located on the oil/water interface.

The composition may be in the form of a cast product, such as a sick in the case of a lipstick or a lip care product. The composition may also be in a variety of other forms, for example a liquid of greater or lesser viscosity, a gel or a paste. The composition may also be in solid form, for example a bar to be moistened for use, to allow it to disintegrate.

The cosmetic composition can be a makeup composition, including a lipstick, a liquid gloss, a lipstick paste, a blusher, a lip crayon, a solid or fluid foundation, a concealer or eye contour product, an eye liner, a mascara, a nail polish, an eye shadow, a body or hair makeup product, or a sun care product, or skin coloring product.

One aspect of the present disclosure is a lipstick, which may be liquid or solid, comprising a composition as defined above.

Another aspect of the present disclosure is a foundation comprising a composition as defined above.

Yet another aspect of the present disclosure is a nail polish comprising a composition as defined above.

Still yet another aspect of the present disclosure is a mascara comprising a composition as defined above.

Further still another aspect of the present disclosure is a product for coloring hair fibers and comprising a composition as defined above.

An addition aspect of the present disclosure is also a process for making up the skin, the lips, the nails, or hair in which a composition as defined above is applied to the skin, the lips, the nails, or hair.

Throughout the description, the term “comprising a” should be understood as being synonymous with “comprising at least one” unless specified to the contrary.

The ranges given include the limit values, unless stated to the contrary.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific example are reported as precisely as possible. Any numerical value, however, inherently contains certain errors necessarily resulting from the standard deviation found in their respective testing measurements.

The following examples are intended to illustrate the present disclosure in a non-limiting manner.

### EXAMPLES

Cosmetic compositions comprising at least one composite pigment with the following formulations were produced. The compositions were prepared using conventional cosmetic preparation methods.

### Example 1

### Lipstick

A lipstick having the following composition was prepared (quantities expressed in percentage by weight relative to the total weight of the composition):

| | |
|---|---|
| Polyethylene wax (Polywax 500 from BARECO) | 8.8% |
| Microcrystalline wax (SP 18 from STRAHL & | 4% |
| PITSCH) | |
| Palmitic-lauric-stearic acid triglyceride | 5% |
| (Softisan 100 from SASOL) | |
| Octyldodecanol | 17.5% |
| Lanolin oil | 10.7% |
| Acetylated lanolin oil | 10.7% |
| Isopropyl lanolate | 10.7% |
| Tridecyl trimellilate | 11.7% |
| Diisotearyl malate | 14.6% |
| Phenyl trimethicone (DC 556 from DOW CORNING) | 4.8% |
| Silica D&C Red No. 7 composite pigment¹ | 1.5% |

¹Composite pigment comprises 50 parts by weight of D&C Red No. 7 organic pigment per 100 parts of an inorganic core of silica having a mean size of 15 nm and a specific surface area of 200 m²/g and made using a polymethylhydrogen siloxane binder.

### Example 2

### Lipstick

A lipstick having the following composition was prepared (quantities expressed in percentage by weight relative to the total weight of the composition):

| | |
|---|---|
| Polyethylene wax (Polywax 500 from BARECO) | 8.8% |
| Microcrystalline wax (SP 18 from STRAHL & | 4% |
| PITSCH) | |
| Palmitic-lauric-stearic acid triglyceride | 5% |
| (Softisan 100 from SASOL) | |
| Octyldodecanol | 17.5% |
| Lanolin oil | 10.7% |
| Acetylated lanolin oil | 10.7% |
| Isopropyl lanolate | 10.7% |
| Tridecyl trimellilate | 11.7% |
| Diisotearyl malate | 14.6% |
| Phenyl trimethicone (DC 556 from DOW CORNING) | 4.8% |
| TiO₂/D&C Red No. 7 composite pigment² | 1.5% |

²Composite pigment comprises 50 parts by weight of D&C Red No. 7 organic pigment per 100 parts of an inorganic core of silica having a mean size of 20 nm and a specific surface area of 50 m²/g, this composite pigment being made using a polymethylhydrogen siloxane binder.

### Comparative Example

### Lipstick

A lipstick was prepared having the following composition, not in accordance with the present disclosure since it was made with only a conventional pure organic pigment as its pigment (quantities expressed in percentage by weight relative to the total weight of the composition):

| | |
|---|---|
| Polyethylene wax (Polywax 500 from BARECO) | 8.8% |
| Microcrystalline wax (SP 18 from STRAHL & | 4% |
| PITSCH) | |
| Palmitic-lauric-stearic acid triglyceride | 5% |
| (Softisan 100 from SASOL) | |
| Octyldodecanol | 17.5% |
| Lanolin oil | 10.7% |
| Acetylated lanolin oil | 10.7% |
| Isopropyl lanolate | 10.7% |
| Tridecyl trimellilate | 11.7% |
| Diisotearyl malate | 14.6% |
| Phenyl trimethicone (DC 556 from DOW CORNING) | 4.8% |
| Pure D&C No. 7 organic pigment³ | 1.5% |

³D&C Red No. 7 organic pigment.

The compositions of Examples 1, 2 and of the Comparative Example had the same ingredients in the same proportions, apart from the pigments.

In order to prepare each of the compositions of Examples 1 and 2, and of the Comparative Example, the pigment was ground in the presence of a fraction of the oils. The remaining oil was mixed with the waxes and heated to about 90° C. After being homogenized, the ground pigment was added thereto. The mixture was then cast in molds and cooled. The lightness L* and the saturation C* in the CIE L*a*b* color space were measured for each of the compositions of Examples 1, 2, and of the Comparative Example.

| | | | Comparative |
|---|---|---|---|
| | Example 1 | Example 2 | Example |
| Lightness L* | 34.6 | 36.6 | 34.8 |
| Saturation C* | 39.0 | 41.9 | 39.0 |

The compositions of Examples 1 and 2, and of the Comparative Example were flattened uniformly between two quartz plates, one of which was hollowed out to a thickness of 30 μm. Total transmission was measured ranging from 400 nm to 700 nm using a JASCO V-550 spectrophotometer fitted with an integration sphere.

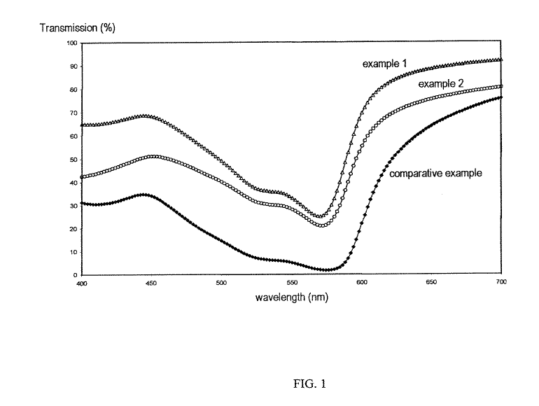

The curves corresponding to the total transmission measurements for the compositions of Examples 1 and 2 and for the Comparative Example are reproduced in FIG. 1.

It can be seen that the compositions of Examples 1 and 2 are less absorbent and therefore more transparent or translucent than the composition of the Comparative Example.

The covering powers of the compositions of Examples 1 and 2 and of the Comparative Example were measured using the method described above.

The measured covering power of the composition of Example 1 was 18, that of the composition of Example 2 was 22, whereas the composition of the Comparative Example presented a measured covering power of 46.

The composition of the Comparative Example is more covering than the compositions of Examples 1 and 2.

### Example 3

### Nail Varnish

A nail varnish can be prepared having the following composition in accordance with the present disclosure (quantities expressed in percentage by weight relative to the total weight of the composition):

| | |
|---|---|
| Nitrocellulose | 19% |
| N-ethyl-o,p-toluene sulfonamide | 6% |
| Acetyl tributyl citrate | 6% |
| Rheological agent (hectorite) | 1.2% |
| Silica/D&C Red No. 7 composite pigment | 3% |
| Isopropanol | 8% |
| Ethyl acetate/butyl acetate | qsp 100 |

This composition is transparent and presents a saturated color.

What is claimed is:

1. A composition comprising particles of at least one composite pigment, said particles comprising an inorganic core which is at least partially coated with at least one organic coloring substance,
wherein the at least partially coated inorganic core is present in an amount sufficient for the saturation C* of the composition to range from 25 to 100, and
wherein the composition has a covering power ranging from 1 to 25.

2. The composition according to claim 1, wherein the at least one composite pigment is present in the composition in an amount ranging from 0.05% to 10% by weight, relative to the total composition weight.

3. The composition according to claim 2, wherein the at least one composite pigment is present in the composition in an amount ranging from 0.1% to 8% by weight, relative to the total composition weight.

4. The composition according to claim 3, wherein the at least one composite pigment is present in the composition in an amount ranging from 0.1% to 5% by weight, relative to the total composition weight.

5. The composition according to claim 4, wherein the at least one composite pigment is present in the composition in an amount ranging from 0.1% to 3% by weight, relative to the total composition weight.

6. The composition according to claim 5, wherein the at least one composite pigment is present in the composition in an amount ranging from 0.5% to 3% by weight, relative to the total composition weight.

7. The composition according to claim 1, wherein the at least one composite pigment comprises the at least partially coated inorganic core in an amount sufficient for the saturation C* of the composition to range from 30 to 100.

8. The composition according to claim 1, wherein the covering power ranges from 5 to 24.9.

9. The composition according to claim 8, wherein the covering power ranges from 10 to 24.9.

10. The composition according to claim 9, wherein the covering power ranges from 15 to 24.9.

11. The composition according to claim 1, wherein the at least one organic coloring substance comprises at least one organic pigment.

12. The composition according to claim 1, wherein the mean size of the inorganic core ranges from 1 nm to 100 nm.

13. The composition according to claim 12, wherein the mean size of the inorganic core ranges from 5 nm to 75 nm.

14. The composition according to claim 13, wherein the mean size of the inorganic core ranges from 10 nm to 50 nm.

15. The composition according to claim 1, wherein the specific surface area of the inorganic core ranges from 1 m²/g to 1,000 m²/g.

16. The composition according to claim 15, wherein the specific surface area of the inorganic core ranges from 10 m²/g to 600 m²/g.

17. The composition according to claim 16, wherein the specific surface area of the inorganic core ranges from 20 m²/g to 400 m²/g.

18. The composition according to claim 1, wherein the inorganic core is in a form chosen from spherical, globular, polyhedral, acicular, spindle-shaped, and flattened forms.

19. The composition according to claim 1, wherein the inorganic core comprises at least one material chosen from metallic salts, metal oxides, alumina, glass, ceramics, graphite, silica, silicates, and synthetic micas.

20. The composition according to claim 19, wherein the inorganic core comprises at least one metal oxide chosen from oxides of titanium, zirconium, cerium, zinc, iron, iron blue, chromium, and aluminum.

21. The composition according to claim 20, wherein the at least one metal oxide is chosen from oxides of titanium, iron, cerium, zirconium, zinc, and aluminum.

22. The composition according to claim 21, wherein the inorganic core comprises titanium dioxide.

23. The composition according to claim 19, wherein the inorganic core comprises at least one silicate chosen from aluminosilicate and borosilicate.

24. The composition according to claim 19, wherein the inorganic core comprises silica.

25. The composition according to claim 1, wherein the at least one organic coloring substance is present in an amount ranging from 10 to 500 parts by weight per 100 parts of inorganic core.

26. The composition according to claim 25, wherein the at least one organic coloring substance is present in an amount ranging from 20 to 250 parts by weight per 100 parts of inorganic core.

27. The composition according to claim 26, wherein the at least one organic coloring substance is present in an amount ranging from 40 to 125 parts by weight per 100 parts of inorganic core.

28. The composition according to claim 1, wherein the at least one organic coloring substance is chosen from carmine cochineal, organic pigments of azo, anthraquinone, indigo, xanthene, pyrene, quinoline, triphenylmethane and fluorane dyes, and organic lakes, insoluble sodium, potassium, calcium, barium, aluminum, zirconium, strontium and titanium salt, and acid dyes.

29. The composition according to claim 28, wherein the at least one acid dye is chosen from azo, anthraquinone, indigo, xanthene, pyrene, quinoline, triphenylmethane and fluorane dyes, and other dyes comprising at least one group chosen from carboxylic and sulfonic acid groups.

30. The composition according to claim 1, wherein the at least one organic coloring substance comprises at least one organic pigment chosen from D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6, and organic lakes supported by an organic support comprising at least one colophane and/or aluminum benzoate.

31. The composition according to claim 30, wherein the at least one organic coloring substance comprises at least one organic lake chosen from D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, and FD&C Yellow No. 6 Aluminum lake.

32. The composition according to claim 1, wherein the at least one composite pigment comprises at least one binder contributing to fixing the at least one organic coloring substance on the inorganic core.

33. The composition according to claim 32, wherein the at least one binder comprises at least one compound chosen from silicone compounds, polymeric compounds, oligomeric compounds comprising at least one organosilane, organosilane compounds of a fluoroalkylated organosilane, polysiloxane compounds, and coupling agents.

34. The composition according to claim 33, wherein the at least one binder comprises polymethylhydrogen siloxane.

35. The composition according to claim 33, wherein the at least one coupling agent is chosen from those based on a silane, a titanate, an aluminate and/or a zirconate.

36. The composition according to claim 33, wherein the at least one binder comprises at least one silicone compound.

37. The composition according to claim 1, wherein the inorganic core is colored.

38. The composition according to claim 1, wherein the composition does not comprise uncoated particles of titanium dioxide.

39. The composition according to claim 1, further comprising at least one cosmetically and/or dermatologically active ingredient.

40. The composition according to claim 1, further comprising at least one additive chosen from fatty bodies, waxes, rubber, and film-forming polymers.

41. The composition according to claim 1, comprising at least one additional coloring substance that is different from the at least one composite pigment.

42. The composition according to claim 41, wherein the at least one additional coloring substance that is different from the at least one composite pigment is chosen from mineral pigments, organic pigments, pearlescent pigments, liposoluble colorings, and hydrosoluble colorings.

43. The composition according to claim 1, wherein the composition is in solid form.

44. The composition according to claim 1, wherein the composition is in liquid, paste, or gel form.

45. The composition according to claim 1, wherein the at least one organic coloring substance is not melanin.

46. The composition according to claim 1, wherein the at least one composite pigment is not an interference pigment.

47. The composition according to claim 1, wherein the saturation C* of the at least one composite pigment is greater than 30.

48. The composition according to claim 32, wherein the at least one binder is present in an amount less than or equal to 5% by weight, relative to the total weight of the at least one composite pigment.

49. The composition according to claim 32, wherein the at least one binder is organic.

50. The composition according to claim 1, wherein the mean gloss T₀h of the composition is greater than or equal to 30.

51. The composition according to claim 50, wherein the mean gloss T₀h of the composition is greater than or equal to 40

52. The composition according to claim 51, wherein the mean gloss T₀h of the composition is greater than or equal to 45.

53. The composition according to claim 52, wherein the mean gloss T₀h of the composition is greater than or equal to 50.

54. The composition according to claim 53, wherein the mean gloss T₀h of the composition is greater than or equal to 60.

55. The composition according to claim 54, wherein the mean gloss T₀h of the composition is greater than or equal to 70.

56. The composition according to claim 1, wherein the composition is a liquid.

57. The composition according to claim 1, wherein the composition is a lipstick.

58. The composition according to claim 1, wherein the composition is a foundation.

59. The composition according to claim 1, wherein the composition is a nail polish.

60. The composition according to claim 1, wherein the composition is a mascara.

61. The composition according to claim 1, wherein the composition is a product for coloring hair fibers.

62. The composition according to claim 1, wherein the particulate amount Q of the composition is greater than or equal to 5%.

63. The composition according to claim 62, wherein the particulate amount Q of the composition is greater than or equal to 7.5%.

64. The composition according to claim 1, wherein the particulate amount Q of the composition is greater than or equal to 10%.

65. The composition of claim 1, wherein the composition comprises an oil having a molar mass ranging from 650 g/mol to 10,000 g/mol.
